(19) United States
(12) Patent Application Publication
(10) Pub. No.: US 2008/0015516 A1
(43) Pub. Date: Jan. 17, 2008

(54) Microneedle Devices With Controlled Uncapping

(75) Inventor: Gilad Lavi, Rishon Lezion (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: NANOPASS TECHNOLOGIES LTD., Nes Ziona (IL)

(21) Appl. No.: 11/776,580

(22) Filed: Jul. 12, 2007

Related U.S. Application Data
(60) Provisional application No. 60/807,073, filed on Jul. 12, 2006.

(51) Int. Cl. A61M 5/158

(52) U.S. Cl. 604/263

(57) ABSTRACT

A microneedle device includes a device body supporting a number of microneedles. A protective cover assumes a closed state in which it protects the microneedles against inadvertent contact and an open state in which the microneedles are exposed. The device body and the protective cover are configured such that, for at least part of a motion from the closed state towards the open state, the protective cover is guided by mechanical engagement with the device body configured to prevent impact between the protective cover and the microneedles, at least until the protective cover has cleared an impact risk region around the microneedles.

### FIELD AND BACKGROUND OF THE INVENTION

The present invention relates to microneedle devices and, in particular, it concerns microneedle devices with controlled uncapping.

Microneedles are known for a wide range of applications including, but not limited to, transdermal and intradermal drug delivery, cosmetic and diagnostic applications. Microneedles or micro-protrusions can also be used for pretreatment of skin by breaking the outer layer of the skin. Microneedles are, by definition, small, typically less than 1 mm high, and frequently in the range of 500 microns down to a few tens of microns. Some microneedles are made sharp so as to enable efficient penetration into the epidermis.

A range of materials and processes have been suggested in the art for fabrication of microneedles. Non-limiting examples of materials include silicon and silicon dioxide, metals, metal alloys, polymers, and glass.

While microneedles might be robust enough to perform their intended function, such as penetration of a biological barrier (for example, the skin or mucous membranes), they could be damaged by contact with any hard surface prior to use, have their sterility compromised. For this reason) it is important to provide a protective cover for the device prior to use.

Removal of this cover prior to use presents a particularly pronounced risk of damage to the microneedles. In order to prevent accidental removal of the cover, the device is designed to require a certain threshold of force to remove the cover. There is a natural reflex for many people, when overcoming a resistance to separate two objects and suddenly encountering reduced resistance, to overcompensate and cause a “rebound” motion in which the hands move back towards each other. In the context of a conventional needle, this is not generally problematic since the cap remains engaged on the length of the needle and no damage is usually done by the rebound action. In microneedle devices, on the other hand, the rebound motion typically occurs while the edges of the cover are in close proximity to the fine microneedles. It is not uncommon for a rebound motion during removal of the cover during uncapping to result in a collision of the cap with the microneedles which may damage the microneedles.

It should be noted that, again unlike conventional needles, due to the small dimensions of the microneedles, it may be difficult for the user to verify whether damage has occurred. This presents a risk of attempted use of a damaged product which may not function as intended, for example, failing to deliver the intended dosage of a drug.

Further, there is a need for a device that allows recapping of the cap onto the microneedles, in order to reduce sharps exposure for the sake of safe disposal (while protecting the person undertaking recapping, during the procedure). While there exist various examples of mechanisms for recapping conventional needles, mostly for a safety purposes, after use and prior to disposal, the current mechanisms and the prior art do not teach a device for safe uncapping of needles or microneedles, nor for re-capping of needles or microneedles combined with uncapping.

There is therefore a need for a microneedle device which would provide a controlled uncapping motion which would prevent damage being caused to the microneedles during uncapping. It would also be advantageous if the device provided for safe recapping of the device after use prior to disposal.

### SUMMARY OF THE INVENTION

The present invention is a microneedle device which provides controlled uncapping motion.

According to the teachings of the present invention there is provided, a microneedle device comprising: (a) a device body including a microneedle substrate surface; (b) at least one microneedle projecting from the microneedle substrate surface; and (c) a protective cover deployable between a closed state in which the protective cover protects the at least one microneedle against inadvertent contact and an open state in which the at least one microneedle is exposed to facilitate bringing the at least one microneedle into functional engagement with a surface, wherein the device body and the protective cover are configured such that, for at least part of a motion from the closed state towards the open state, the protective cover is guided by mechanical engagement with the device body configured to prevent impact between the protective cover and the at least one microneedle at least until the protective cover has cleared an impact risk region around the at least one microneedle.

According to a further feature of the present invention, the device body is an elongated body having a length greater than each of two lateral dimensions, the microneedle substrate surface being located at an end portion of the elongated body, and wherein the protective cover includes a resilient clip configured to resiliently deform during motion from the closed state towards the open state.

According to a further feature of the present invention, at least part of the device body has a generally round cross-sectional shape, and wherein the resilient clip is configured to circumscribe more than 180 degrees and less than 360 degrees, and preferably between about 225 degrees and about 315 degrees, around the device body when in the closed state.

According to a further feature of the present invention, the mechanical engagement is generated at least in part by a projection from the device body engaged within a corresponding opening formed in the protective cover.

According to a further feature of the present invention, the projection defines a direction of projection, and wherein the projection and the corresponding opening are configured to inhibit rotation of the protective cover relative to the device body about the direction of projection.

According to a further feature of the present invention, a cross-section taken near a base of the projection has a major dimension parallel to the length and a minor dimension perpendicular to the length.

According to a further feature of the present invention, the projection includes an overhanging portion extending in a direction away from the microneedle substrate surface, engagement of the corresponding opening with the overhanging portion delimiting a rotational motion of the protective cover away from the impact risk region.

According to a further feature of the present invention, the mechanical engagement between the protective cover and the device body defines an axis of rotation for at least an initial part of a motion of the protective cover from the closed state towards the open state.

According to a further feature of the present invention, the mechanical engagement is configured to allow translational displacement of the protective cover only after rotation through a predefined angle from the closed state.

According to a further feature of the present invention, the mechanical engagement is further configured to define a path of the translational displacement.

According to a further feature of the present invention, the mechanical engagement comprises a permanent hinged interconnection between the protective cover and the device body about the axis of rotation.

According to a further feature of the present invention, there is also provided a recapping lock mechanism configured to allow displacement of the protective cover once from the closed state to the open state and, after returning to the closed state, to lock so as to prevent subsequent displacement to the open state.

According to a further feature of the present invention, a normal to a plane of the microneedle substrate surface defines a reference direction, and wherein the mechanical engagement defines a direction of linear displacement substantially transverse relative to the reference direction.

According to a further feature of the present invention, the at least one microneedle is implemented as a linear array of microneedles.

According to a further feature of the present invention, the linear array is deployed parallel to, and substantially adjacent to, an edge of the device body.

There is also provided according to a further feature of the present invention, a microneedle device comprising: (a) an elongated device body having a length greater than each of two lateral dimensions, the device body including a microneedle substrate surface located at an end portion of the device body; (b) at least one microneedle projecting from the microneedle substrate surface; and (c) a protective cover deployable between a closed state in which the protective cover protects the at least one microneedle against inadvertent contact and an open state in which the at least one microneedle is exposed to facilitate bringing the at least one microneedle into functional engagement with a surface, the protective cover including a resilient clip configured to resiliently deform to allow motion from the closed state towards the open state in a direction non-parallel to the length.

According to a further feature of the present invention, at least part of the device body has a generally round cross-sectional shape, and wherein the resilient clip is configured to circumscribe more than 180 degrees and less than 360 degrees, and preferably between about 225 degrees and about 315 degrees, around the device body when in the closed state.

According to a further feature of the present invention, the device body and the protective cover are configured such that, for at least part of a motion from the closed state towards the open state, the protective cover is guided by mechanical engagement with the device body configured to prevent impact between the protective cover and the at least one microneedle at least until the protective cover has cleared an impact risk region around the at least one microneedle.

According to a further feature of the present invention, the device body includes a lateral projection and the protective cover features a corresponding opening, the corresponding opening being engaged with the lateral projection in the closed state so as to at least partially delimit a path of motion from the closed state towards the open state.

According to a further feature of the present invention, the projection defines a direction of projection, and wherein the projection and the corresponding opening are configured to inhibit rotation of the protective cover relative to the device body about the direction of projection.

According to a further feature of the present invention, a cross-section taken near a base of the projection has a major dimension parallel to the length and a minor dimension perpendicular to the length.

According to a further feature of the present invention, the projection includes an overhanging portion extending in a direction away from the microneedle substrate surface, engagement of the corresponding opening with the overhanging portion delimiting a rotational motion of the protective cover away from the microneedle substrate surface.

There is also provided according to the teachings of the present invention, a method of using a microneedle device comprising the steps of: (a) providing a microneedle device including: (i) an elongated device body having a length greater than each of two lateral dimensions, the body including a microneedle substrate surface located at an end portion of the elongated body, (ii) at least one microneedle projecting from the microneedle substrate surface, and (iii) a protective cover deployed so as to protect the at least one microneedle against inadvertent contact; and (b) displacing the protective cover so as to expose the at least one microneedle for functional engagement with a surface, wherein the displacing is performed in a direction non-parallel to the length.

According to a further feature of the present invention, the displacing is performed in a direction generally transverse to the length.

According to a further feature of the present invention, the displacing is performed in a generally pivotal motion.

According to a further feature of the present invention, device body and the protective cover have features for mechanical engagement such that at least an initial part of the displacing occurs along a path of motion delineated by the features for mechanical engagement.

### BRIEF DESCRIPTION OF THE DRAWINGS

The invention is herein described, by way of example only, with reference to the accompanying drawings, wherein:

FIG. 1 is an isometric view of a first embodiment of a microneedle device, constructed and operative according to the teachings of the present invention, combining linear and rotational movements for cover removal.

FIG. 2 is an isometric view of the embodiment of FIG. 1, after attachment of a syringe and completion of a first stage of the uncapping motion, including a rotational movement.

FIG. 3 is an isometric view of the embodiment of FIG. 1 after completion of a second stage of the uncapping motion, including a linear movement in a first direction.

FIG. 4 is an isometric view of the embodiment of FIG. 1 after completion of a third stage of the uncapping motion, including removal of the cover by movement in a second direction.

FIGS. 5A-5C are enlarged partial isometric views illustrating an interlocking configuration delimiting the sequence of motion of the cover in the embodiment of FIG. 1.

FIG. 6A and FIG. 6B illustrate a second embodiment of a microneedle device, constructed and operative according to the teachings of the present invention, in which a cover is removed by linear movement. FIG. 6A shows the device before removal of the cover while FIG. 6B illustrates the same device after removal of the cover.

FIG. 7 is a cross-sectional view taken through the device of FIG. 6A.

FIGS. 8A-8C are isometric views of a third embodiment of a microneedle device, constructed and operative according to the teachings of the present invention, having a single use covering and locking system. FIG. 5A illustrates the embodiment before use, FIG. 8B illustrates the same embodiment during use and FIG. 8C shows it in its recapped locked position, ready to be discarded.

FIG. 9 is an isometric exploded view of the embodiment of FIG. 8A.

FIG. 10 is a side view of the embodiment of FIG. 8A.

FIGS. 11A-11C are cross-sectional views of the embodiment in FIG. 10 taken along line A-A. FIG. 11A shows the device before use, FIG. 11B shows the device after uncapping ready for use and FIG. 11C shows the device in its recapped locked state, ready to be discarded.

FIG. 12 is a front view of the embodiment of FIG. 8A.

FIGS. 13A-13B are cross-sectional views taken along the line B-B in FIG. 12. FIG. 13A illustrates the device before use, and FIG. 13B illustrates the device during use.

FIG. 14 is a further front view of the embodiment of FIG. 8A.

FIGS. 15A-15C are cross-sectional views taken along the line C-C in FIG. 14. FIG. 15A illustrates the embodiment before use, FIG. 15B illustrates the same during use and FIG. 15C illustrates the device in a locked position ready to discard.

FIGS. 16A-16C are partial isometric cross-sectional views of the embodiment of FIG. 8A. FIG. 16A illustrates the embodiment before use, FIG. 16B illustrates the same during use and FIG. 16C illustrates the device in its locked position ready to discard.

FIG. 17 is an isometric view of a fourth embodiment of a microneedle device, constructed and operative according to the teachings of the present invention, attached to a syringe.

FIG. 18 is an enlarged isometric view of the embodiment of FIG. 17 with the cover in place.

FIG. 19 is a view similar to FIG. 18 after removal of the cover.

FIGS. 20A-20C are a series of isometric views of the embodiment of FIG. 17 showing movement of the cover during uncapping.

FIGS. 21A-21C are a series of partially cut-away isometric views of the embodiment of FIG. 17 showing movement of the cover during uncapping.

FIGS. 22A and 22B are enlarged views of parts of FIGS. 20A and 21A, respectively.

FIG. 23 is an isometric view of a fifth embodiment of a microneedle device, constructed and operative according to the teachings of the present invention.

FIG. 24 is an isometric view of the cover from the microneedle device of FIG. 23 after removal.

FIG. 25 is a partially cut-away side view of the microneedle device of FIG. 23.

### DESCRIPTION OF THE PREFERRED EMBODIMENTS

The present invention is a microneedle device with controlled uncapping and a corresponding method.

The principles and operation of microneedle devices according to the present invention may be better understood with reference to the drawings and the accompanying description.

### Overview

By way of introduction, the present invention provides microneedle devices in which the microneedles are protected by a cover prior to use and the cover is removed in a manner which protects against the aforementioned problems of damage to the microneedles by impact of the cover on the microneedles during removal of the cover. The invention will be illustrated herein with reference to five non-limiting embodiments. The first four embodiments all provide mechanical engagement between the cover and the body of the device in such a manner as to delimit at least part of the path of motion for removing the cover, thereby preventing collision of the cover with the microneedles. The fifth embodiment provides a cover which is configured to allow removal in a lateral direction and relies upon the user to do so. Additionally, various of the embodiments illustrate arrangements which facilitate safe recapping and/or locking on recapping to prevent inadvertent re-use.

Thus, in general terms, according to a first aspect of the present invention, there is provided a microneedle device, constructed and operative according to the teachings of the present invention, having a device body including a microneedle substrate surface, and one or more microneedles projecting from the microneedle substrate surface. A protective cover is deployable between a closed state in which it protects the microneedles against inadvertent contact and an open state in which the microneedles are exposed to facilitate bringing the microneedles into functional engagement with a surface, such as a biological barrier. The device body and the protective cover are configured such that, for at least part of a motion from the closed state towards the open state, the protective cover is guided by mechanical engagement with the device body so as to prevent impact between the protective cover and the microneedles, at least until the protective cover has cleared an impact risk region around the microneedles.

At this stage, it will already be clear that this aspect of the present invention provides profound advantages over conventional capping arrangements for microneedle devices. Specifically, by delineating at least an initial part of the path of motion of the cover during uncapping, the motion is controlled in a manner which prevents, under a wide range of normal operating conditions, collision between the cap and the fine microneedles. Thus the aforementioned problems of inadvertent use of a damaged product, as well as contamination, are avoided. This and other advantages of the present invention will become clearer from the following detailed description.

### Definitions

Before addressing the features of the various embodiments in more detail, it will first be helpful to define various terminology as used herein in the description and claims. Firstly, the term “microneedle” is used herein to refer to any structure projecting from an underlying surface to a height no more than 1 millimeter, and typically, between 10 microns and 750 microns. The microneedles may be solid (sometimes referred to as “micro-protrusions”), hollow or otherwise channeled or porous. The microneedles may be formed from any suitable material including, but not limited to, silicon and silicon dioxide, metals, metal alloys, polymers, glass and combinations thereof. Most typically, the microneedles have a penetrating point. In certain particularly preferred implementations, a microneedle structure as taught by U.S. Pat. No. 6,533,949, hereby incorporated by reference, are used. The underlying material from which the microneedles project is referred to interchangeably as the “substrate” or “chip”, independent of the materials and technology through which it is produced.

It should be noted that the “microneedle device” of the present invention may be any device which includes microneedles, for any application and of any type. Furthermore, the device may be a stand-alone device or may be an adapter for use together with another device. Types of device encompassed by the present invention include, but are not limited to, therapeutic, aesthetic medicine, cosmetic and diagnostic devices for drug delivery, fluid sampling and surface treatments, for example, micro-dermabrasion. Certain devices may perform a plurality of these functions, such as sampling and drug delivery in closed loop systems. According to a further innovation of the present invention, of importance in its own right, microneedle devices have been found to be highly advantageous for drug delivery and other applications through mucous membranes, such as the gums and other tissue of the oral cavity. By way of one particularly preferred but non-limiting example, the various embodiments of the present invention will be illustrated herein in the context of drug delivery devices, and more particularly, as a microneedle adapter for use with a conventional syringe for drug delivery.

More specifically, the present invention is illustrated herein in an embodiment having a linear array of microneedles deployed parallel to, and substantially adjacent to, an edge of the device body. Further details of this particularly preferred configuration and its advantages may be found in US Patent Application Publication No. 2005/0209566, which is hereby incorporated by reference.

The term “device body” is used to refer to any element or combination of elements which provides structural support for one or more microneedles. Reference is made in certain embodiments to an “elongated body.” In this context, the term “elongated” is used to refer to a body having a major dimension referred to as “length” and two dimensions orthogonal to the length and to each other (referred to as “lateral dimensions”) which are no more than half the length.

Various directions are defined herein in relation to the “length” dimension. For the purpose of the description and claims, elements or directions appearing more or less parallel to the length are referred to as parallel to the length, even if they differ measurably from accurate parallelism. Conversely, directions referred to as non-parallel are such that the divergence from parallelism is readily apparent, for example, in excess of about 15 degrees. Directions referred to as “transverse” or “lateral” relative to the length or another direction should be assumed to be within 30 degrees on either side of the perpendicular to the direction referred to, i.e., inclined at between about 60 degrees and about 120 degrees to the length or other reference direction.

The closed state of the protective covers of the present invention are referred to as “protecting against inadvertent contact” with the microneedles. It should be noted that the “protection” required is only protection from inadvertent or otherwise unintentional damage. The cover is not typically required to prevent damage to the microneedles from narrow or sharp instruments which could be inserted through narrow gaps between the device body and the protective cover, or through excessive force applied on the cover itself. Geometrically, a cover is considered to protect against inadvertent contact if, alone or together with the other surfaces of the device body, the cover prevents the microneedles from coming into contact with a flat surface in all orientations of the device and the cover prevents objects of width greater than about 8 millimeters (such as a finger) from touching the needles. Optionally, part or all of the protective cover may be formed from transparent material in order to render the microneedle chip visible.

Reference is made to an “open state” of the cover in which the microneedles are exposed to facilitate bringing the microneedles into functional engagement with a surface. The “open state” is defined herein by the lack of an obstruction to bringing the microneedles into engagement with a surface, but does not necessarily uniquely define a position of the protective cover in space. Thus, in certain embodiments, the “open position” is completely detached from the device body such that the device cover can be put in any convenient location. In such cases, motion “towards the open state” is defined as motion from the closed state towards the location or position in which detachment of the cover can occur.

The phrase “functional engagement with a surface” is used to refer to engagement which allows the microneedles to perform their intended function, whether fluid injection, diagnostic sampling, mechanical abrasion or some other intended function, all according to the particular application.

Reference is made to an “impact risk region.” This phrase is used to refer to a region around the microneedles within which unrestricted movements of a magnitude commonly occurring unintentionally during the uncapping process would produce a risk of impact with the microneedles. Typically, this region may be assumed to end about 3 centimeters away from the microneedles. It is important to note that the impact risk region is primarily directly in front of the microneedles and immediately to their side. A protective cover is considered to be outside the impact risk region if any part of the cover is in the geometrical shadow of the device body from the microneedles such that a complex or non-linear motion would be required to cause impact between the cover and the microneedles. Thus, in many cases, the cover may be out of the impact risk region if it is above the device body, even if part of the cover is within 3 centimeters from the microneedles. Furthermore, once the protective cover has moved in a controlled manner to a position considered outside the impact risk region, it can then be released. Any subsequent movement is performed without needing to overcome a threshold of resistance so that it is not considered problematic even if the cover subsequently passes again through the impact risk region, for example, while being removed.

Reference is made to “mechanical engagement” between the protective cover and the device body which at least partially guides motion of the protective cover relative to the device body. Various examples of this mechanical engagement will be illustrated below, ranging from a permanent hinge through sliding bearing surfaces to a simple projection and complementary opening. However, it should be noted that the invention is not limited to these examples, and that the mechanical engagement may take any form within the capabilities of a person having ordinary skill in the art for providing the controlled relative motion required. Examples of suitable forms of mechanical engagement include, but are not limited to, directly abutting surfaces of all shapes, sliding surfaces of all shapes, bearing arrangements, integral hinges, telescopic arrangements, arrangements of lever arms, scissor mechanisms and flexible linkages such as wires, cords or chains. Furthermore, it will be noted that the guidance of motion provided by the mechanical engagement does not need to define a unique path of motion and may instead simply define a limit or envelope to the extent of motion which can occur, so long as it ensures that the protective cover leaves the impact risk region without coming into contact with the microneedles.

With regard to the specific exemplary embodiments, the term “clip” is used to denote any structure resiliently biased to a gripping configuration for gripping part of the device body and temporarily deformed for bringing into engagement so as to grip the device body and for removal therefrom. For simplicity and cost efficiency, the clip preferably employs inherent elastic properties of a material from which it is made, most preferably a resilient polymer material, without requiring any separate spring element. Most preferably, the form of the clip and of the corresponding part of the device body are configured so that the necessary flexing of the cover is induced by pressing the cover towards its closed state or during removal without requiring a separate action to deform the clip.

When reference is made to a “hinge”, this denotes any form of mechanical engagement which defines an axis of relative rotation between the protective cover and the device body. The term “hinge” thus defined includes hinge arrangements with and without a pin element, integral hinges and various other mechanical arrangements of lever arms and the like which define an effective axis of rotation. The term “hinge” may also be used to refer to arrangements where the axis of rotation is not fixed, so long as the motion reasonably approximates to rotation about an axis.

Reference is made to a “direction of motion” of the cover when moving from the closed state towards the open state. When referring to linear translational motion, this direction of motion is intuitively well defined. In the case of arcuate translation or pivotal motion, the “direction of motion” is taken to be the direction in which the center of gravity of the cover moves at the onset of motion, or more technically, a tangent to the path of motion at that point.

In certain embodiments where reference is made to locking of an element to prevent subsequent displacement, it should be appreciated that the “locking” in question is intended to resist non-destructive manually applied opening forces of magnitudes likely to be applied by a user trying to open the device, thereby helping to guard against inadvertent reopening and accidental re-use of a product intended to be for single use only. The device is typically not designed to prevent intentional circumvention of the locking mechanism.

In certain embodiments, at least part of the device body is described as having “a generally round cross-sectional shape.” The phrase “generally round” is used herein to refer to a shape which gives a round or cylindrical visual impression, independent of the presence of various indented or projecting features, or other deviations from a true circular form. Functionally, the part of the device body in question preferably has a sufficient region of surfaces approximating to the round shape to allow sliding on and off of a complementarily shaped clip. In certain particularly preferred embodiments, substantially the entirety of the device body falls within a roughly cylindrical profile, although various parts, particularly in the region of the microneedle interface itself, may vary considerably from a cylindrical shape in order to provide the desired geometrical arrangement.

Finally with regard to definitions, where a clip is described as “circumscribing” a certain angle around a device body, the angle circumscribed refers to the maximum extent around the body through which the clip extends in the closed state, as viewed axially.

### First Embodiment

### Pivot-and-Slide

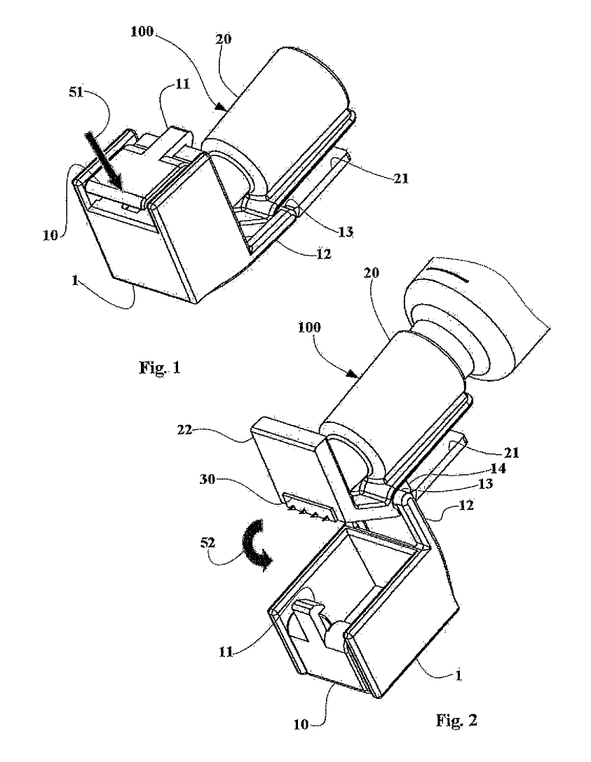

Referring now to FIGS. 1-5C, there is shown a first embodiment of the microneedle device, generally designated 100, constructed and operative according to the teachings of the present invention. This embodiment exemplifies a group of implementations in which the mechanical engagement between the protective cover and the device body defines an axis of rotation for at least an initial part of a motion of the protective cover from the closed state towards the open state. In this particular case, the mechanical engagement is further configured to allow translational displacement of the protective cover only after rotation through a predefined angle from the closed state, and defines a path of the subsequent translational displacement until the cover is released.

Device 100 is here implemented as a syringe adapter, formed with a device body including a main block 20, typically made of plastic, protected by protective cover 1. Protective cover 1 is locked to block 20 at wall 22, to which micro-needle chip 30 is attached. During storage, cover 1 is prevented from undergoing any significant movement relative to micron-needle chip 30. The term “significant” in this context refers to any movement that might allow contact with, or damage to, the micro-needles on the chip.

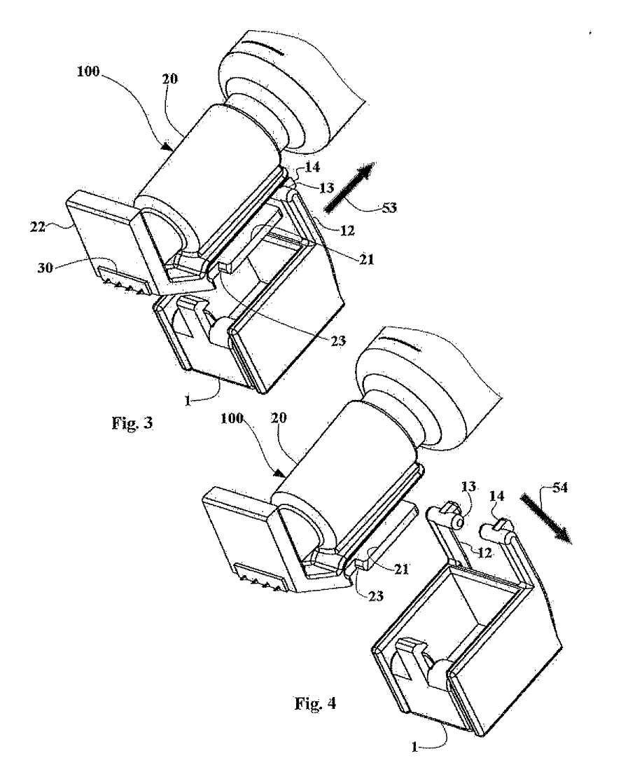

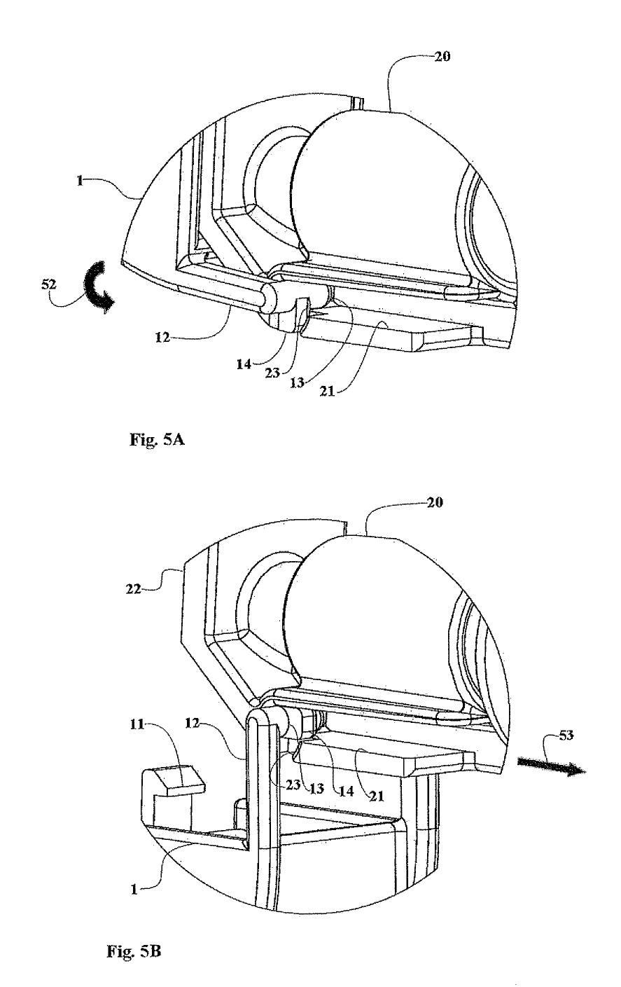

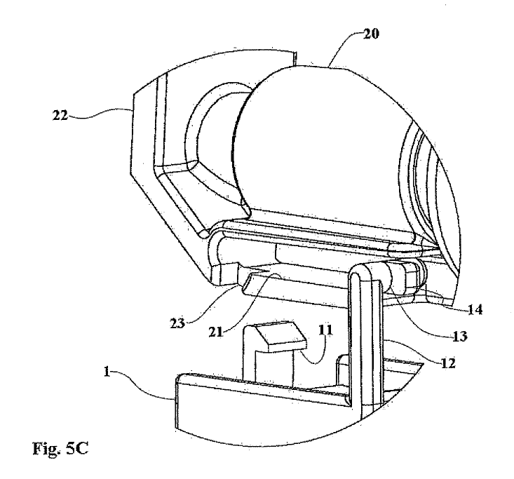

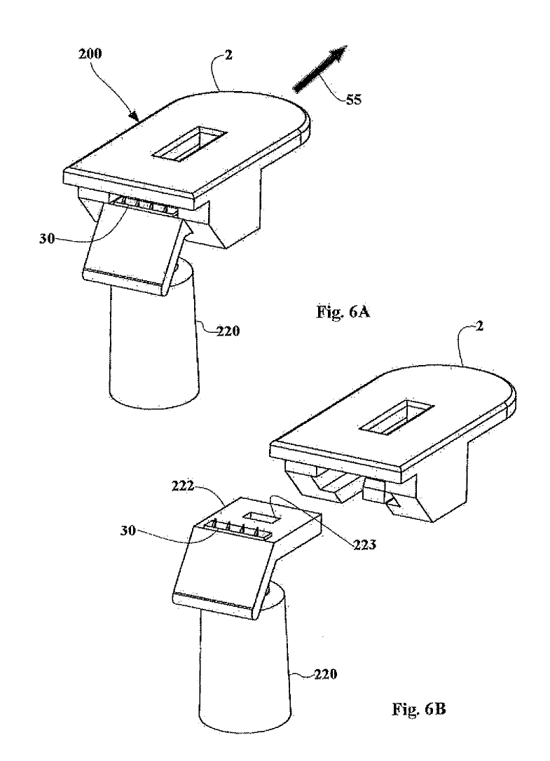

In the particular example illustrated in FIG. 1, cover 1 includes a lever 10 which ends with a catch 11 configured for snap engagement with wall 22. Pressure applied by a finger on lever 10 as illustrated by arrow 51 lifts catch 11, thereby releasing its engagement with wall 22. Cover 1 is then free to undergo pivotal motion about pivot pins 13 as indicated by arrow 52 in FIG. 2. The extent of the rotation is limited by the form of the pivotal mounting, as will be detailed with reference to FIGS. 5A-5C below, so as to stop in the position illustrated in FIG. 2. The cover is then slid in direction 53 so that pivot pin 13 moves along a track 21 until it is released at the end of track 21 as shown in FIG. 3. The pivotal mounting is configured so as to prevent rotation of the cover during this sliding motion so that the cover cannot inadvertently collide with the microneedles. Subsequently, having cleared the impact risk region and being in the geometrical shadow of the device body from the microneedles, protective cover 1 can be moved manually without limitation as illustrated in FIG. 4 by arrow 54.

Details of the pivotal arrangement to delimit the aforementioned sequence of motions are shown in FIGS. 5A-5C, which correspond, respectively, to the states shown in FIGS. 1-3. Pivot pins 13 are supported at the ends of arms 12 and carry asymmetric projections 14 which engage a cut-out bearing surface 23. In the closed position of FIG. 5A, pivot pins 13 are retained at the end of track 21 by abutment of projections 14 and bearing surfaces 23. As the cover is pivoted towards its open state, projections 14 slide across bearing surfaces 23 until they reach the position of FIG. 5B in which they are aligned within track 21. In this state, the parallel upper and lower surfaces of projections 14 slide in contact with the sides of track 21, allowing linear translational displacement but resisting significant pivotal motion until pivot pins 13 clear the end of track 21, as shown in FIG. 5C.

### Second Embodiment

### Transverse Slide

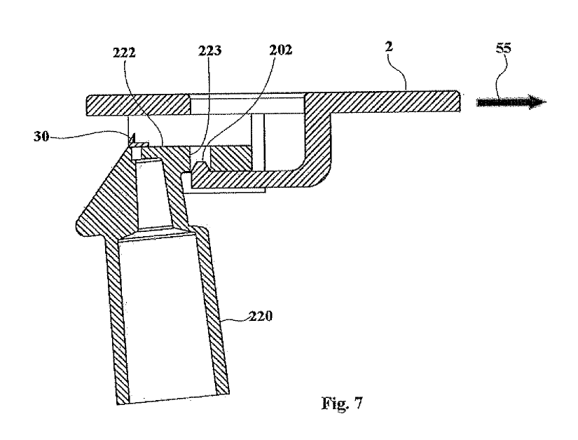

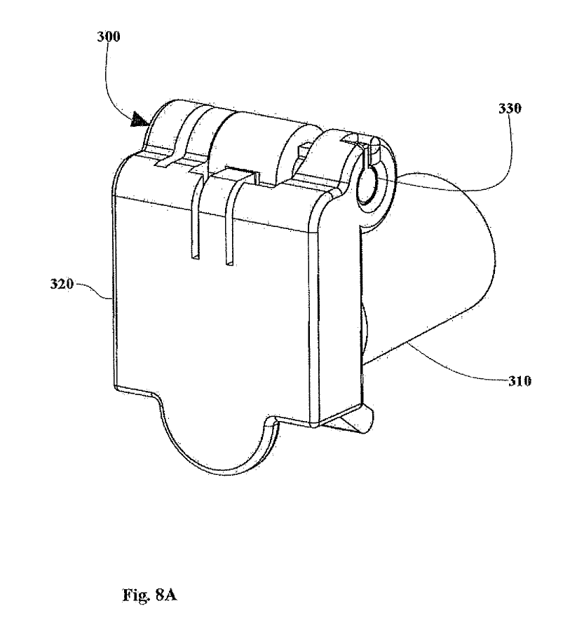

Turning now to FIGS. 6A, 6B and 7, there is shown a second embodiment of a microneedle device, generally designated 200, constructed and operative according to the teachings of the present invention. This embodiment exemplifies a group of implementations in which the mechanical engagement between the protective cover and the device body defines a direction of linear displacement of the cover substantially transverse to a reference direction normal to the plane of the microneedle substrate surface.

Referring now to the drawings, device 200 has a protective cover 2 which is removed in a linear motion along a path designated by arrow 55. The path is here delineated by the edges of wall 222 which also provides the surface to which the microneedle substrate 30 is attached. Protective cover 2 is formed with slots at each side which engage the edges of wall 222 and a central channel which provides clearance to avoid contact with the microneedles. The clearance between the central channel and the microneedles in the closed state is sufficiently small to provide effective protection of the microneedles as defined above, without actually hiding the microneedles from view. Clearly, in this and other cases, a supplementary outer cover (not shown) is typically provided to maintain sterility prior to use, as is standard in the art.

It will be appreciated that the substantially transverse direction of the motion 55 together with the extent of the sliding engagement beyond the region of the microneedles are sufficient to ensure that cover 2 leaves the impact risk region before clearing the engagement with the device body.

Most preferably, device 200 includes retention features for retaining cover 2 in place and to inhibit unintentional removal of the cover prior to use. To this end, as best seen in FIG. 7, protective cover 2 here includes a resilient arm which terminates in a projecting detent 202 configured to engage a corresponding recess 223 in the device body. This engagement inhibits unintentional displacement of the cover until sufficient lateral force is applied to displace detent 202 from recess 223, thus facilitating further displacement of cover 2 until the cover is removed and the microneedles exposed ready for use.

### Third Embodiment

### Hinge with Recapping Lock

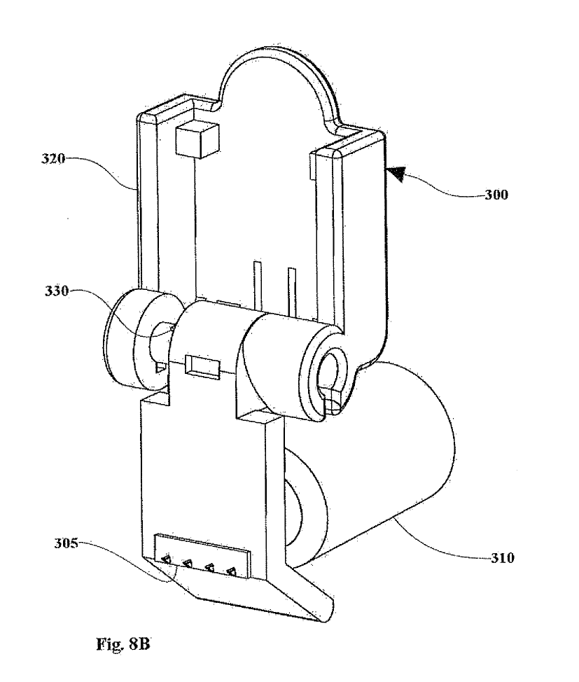

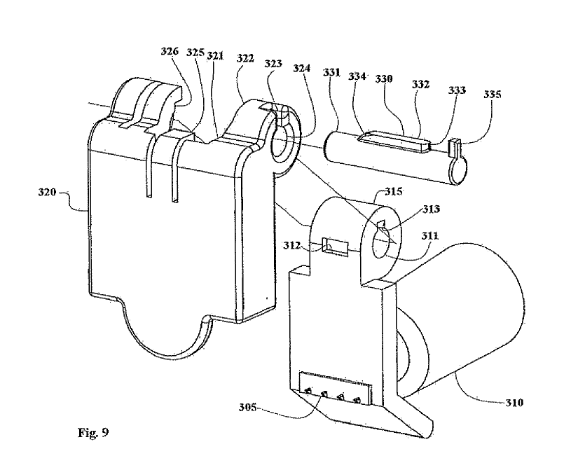

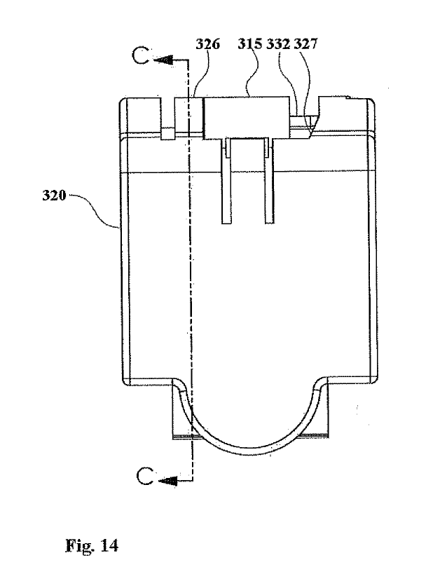

Turning now to FIGS. 8A-16C, there is shown a third embodiment of a microneedle device, constructed and operative according to the teachings of the present invention, generally designated 300. This embodiment exemplifies a group of implementations in which the mechanical engagement between the protective cover and the device body includes a permanent hinged interconnection. It also exemplifies a mechanism, in this case integrated with the hinged interconnection, which allows displacement of the protective cover once from the closed state to the open state and, after returning to the closed state, locks so as to prevent subsequent displacement to the open state. This helps to prevent inadvertent reuse of a disposable single-use device.

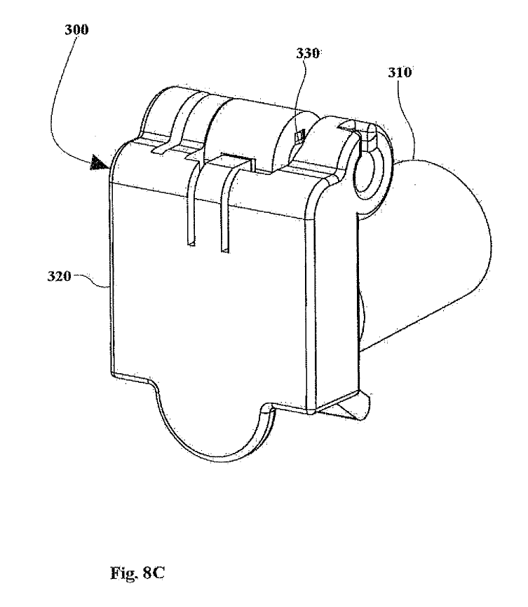

Turning now to the drawings, as best seen in FIG. 9, microneedle device 300 is assembled from three main components: a device body including block 310 which supports microneedle chip 305; a protective cover 320; and a hinge pin 330. Hinge pin 330 features an alignment ridge 332 which engages a corresponding key-hole slot formed by an axial bore 311 and a slot 313 in the hinge-forming part 315 of block 310. This engagement prevents significant relative rotation between the hinge pin 330 and block 310 while allowing axial motion of the hinge pin, as will be detailed below. Hinge pin 330 also features a shear-off projection 335 which serves to provide part of the initial resistance to opening, as well as tamper evident protection.

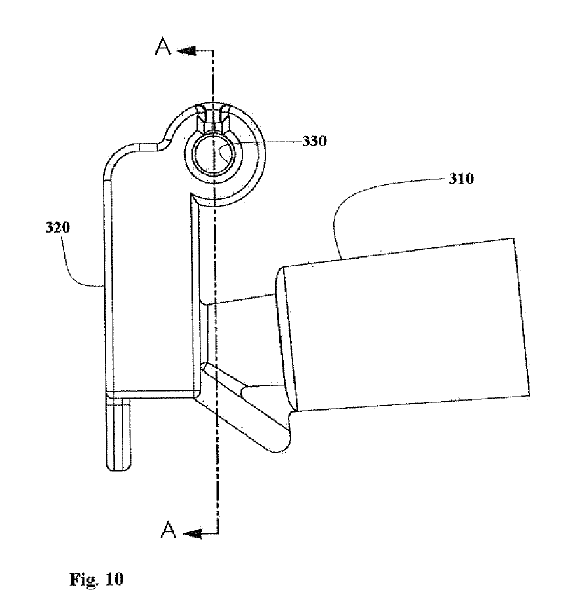

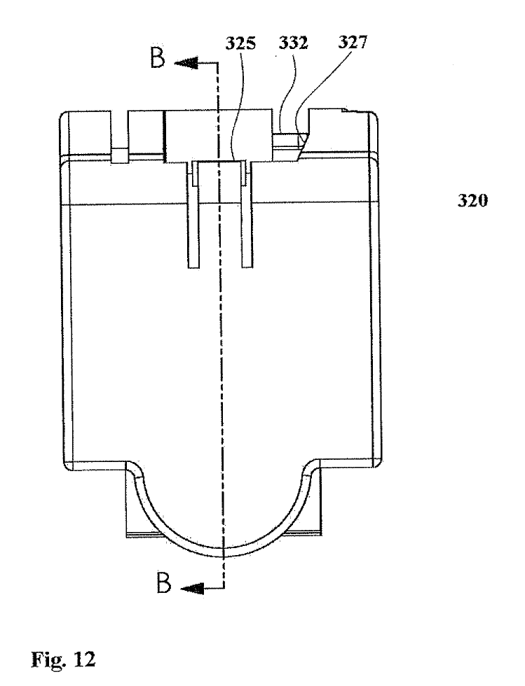

Cover 320 is configured for rotational movement between two defined end positions. In a first position (FIGS. 11A and 13A), the cover is deployed so as to protect the microneedles. In a second position (FIGS. 11B and 13B), the cover is folded up to allow use of the microneedle device. As before, the cover opens in a defined (here rotational) path of motion so as to clear the microneedle region without risk of the cover impacting against the microneedles. A third position, similar to the first, is produced by returning the cover towards its starting position. It is a particularly preferred feature of this embodiment that the device includes a mechanism for preventing re-opening of the cover after it reaches the third position.

In the initial state of FIGS. 5A, 11A, 13A and 15A, shear-off projection 335 is engaged in a corresponding recess 323 (see FIG. 9) in cover 320, thereby providing retention in the initial closed state and evidence that the device has not been used. Additional retention in this position is provided by a resilient element 325 which is biased to engage a recess 312, as best seen in FIGS. 9 and 13A.

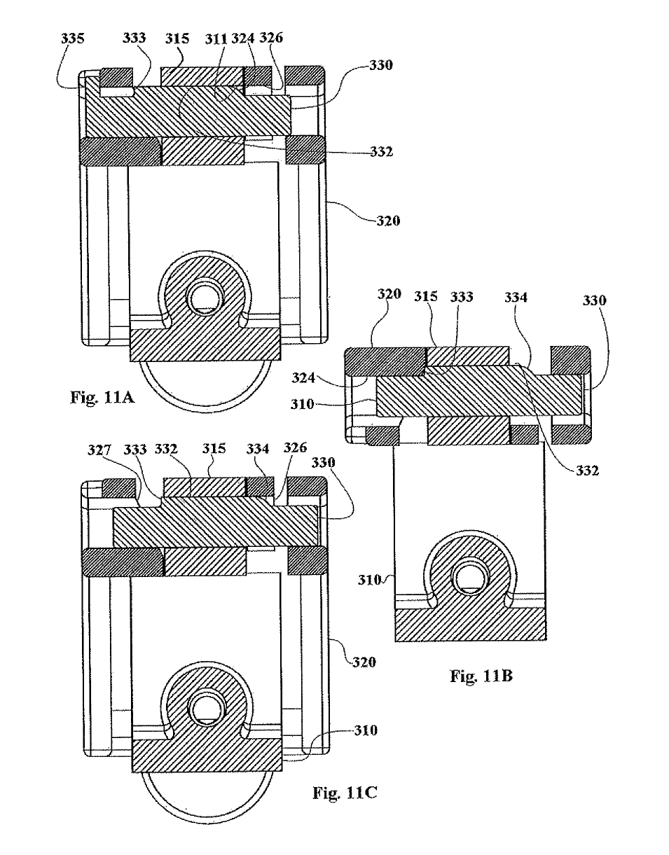

When sufficient force is applied to open cover 320, the rotation of the hinge-forming portion 322 of the cover shears off protrusion 335, and resilient element 325 is displaced from recess 312 as the cover starts to rotate. Hinge pin 330 is also provided with features cooperating with features of the cover 320 and/or block 310 so as to generate axial displacement of the pin during opening of the cover. In the specific case illustrated here, pin 330 is displaced axially by sliding contact with an inclined surface 327 of the cover (see FIG. 12) with an end of alignment ridge 332. This axial motion is best seen by comparing FIGS. 11A and 11B.

Cover 320 is retained in the open state of FIGS. 8B, 13B and 15B by engagement of resilient element 325 in another recess on the rear of hinge-forming part 315 of block 310. In this state, the device is ready for use.

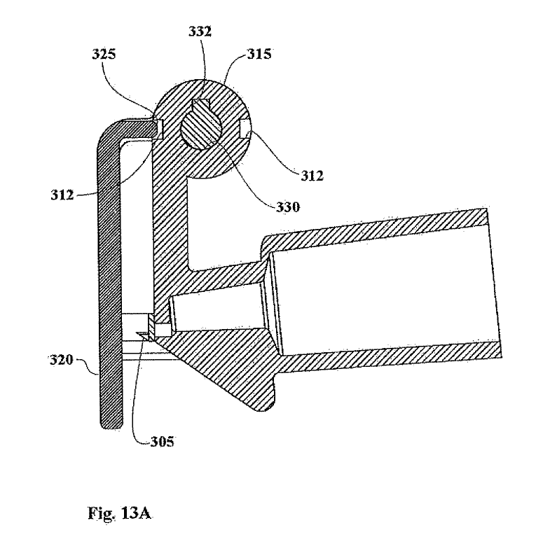

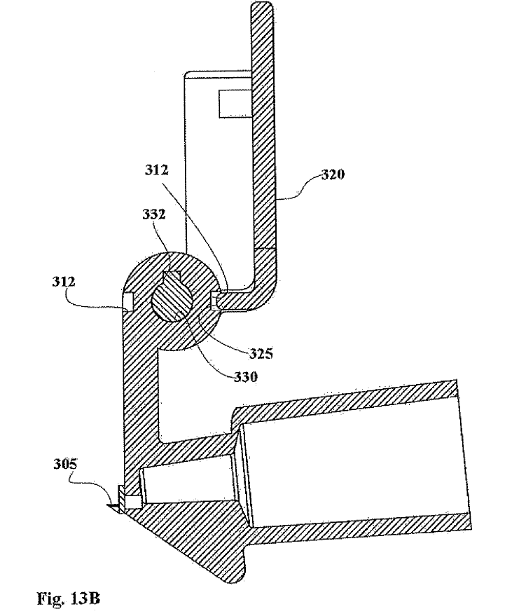

As a result of the axial motion of pin 330, the leading part of alignment ridge 332 extends beyond portion 315 of block 310 as seen in FIG. 11B, thus bringing it into the plane C-C of FIG. 14. A corresponding region of cover 320 is formed with a locking recess 326 as seen in FIGS. 15A-15C. Before use, when alignment ridge 332 has not reached the region of this cross-section (FIG. 15A), locking recess 326 is unobstructed and does not interfere with opening of the cover. After opening of the cover, when hinge pin 330 has migrated axially as described above, it appears within the cross-sectional plane as illustrated in FIG. 15B. As the cover is once again closed, the adjacent region of cover 320 is resiliently deformed to ride over alignment ridge 332 until locking recess 326 becomes aligned with the ridge. At this point, the adjacent region of cover 320 snaps inwards, engaging recess 326 against alignment ridge 332 as shown in FIG. 15C and thus locking the cover against subsequent reopening (at least under normal conditions of manually-applied force not sufficient to break the device).

### Fourth Embodiment

### Side-Release Clip with Guide

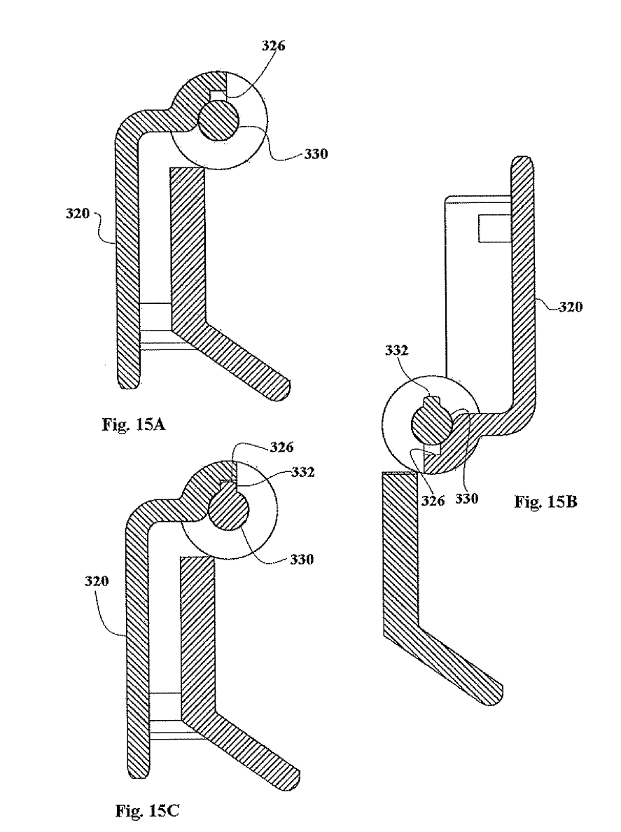

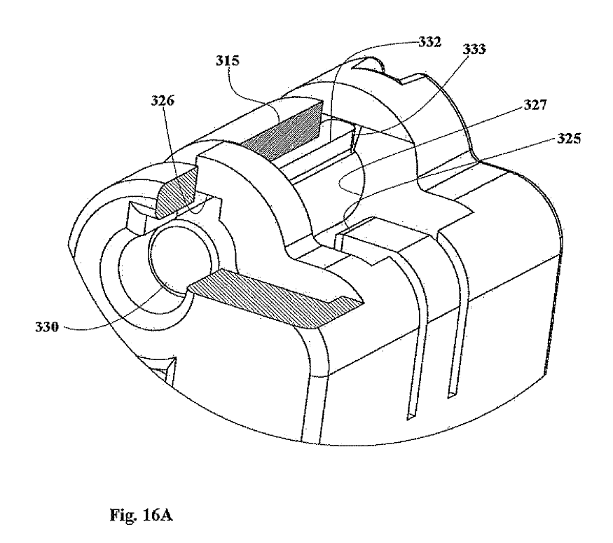

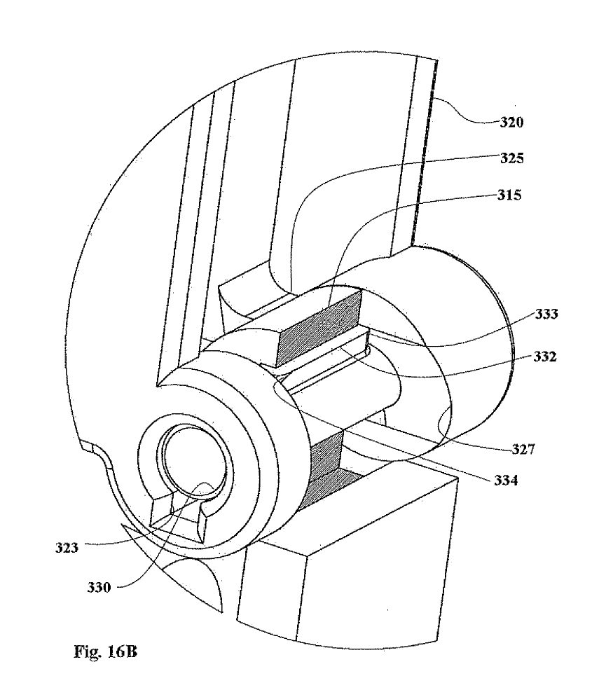

Turning now to FIGS. 17-22B, there is shown a fourth embodiment of a microneedle device, constructed and operative according to the teachings of the present invention, generally designated 400. This embodiment may be regarded in certain respects as a simplified implementation conceptually similar to microneedle device 100 described above. This embodiment additionally illustrates a further aspect of the present invention according to which the protective cover 404 is implemented at least in part as a resilient clip which is deployable from its closed state towards its open state in a direction non-parallel to the length of the device.

The current embodiment is particularly, although not exclusively, of importance in the context of a device with an elongated body 402, i.e., having a length greater than each of two lateral dimensions, and typically at least twice the lateral dimensions, and where the microneedle substrate surface 412 carrying the microneedles is located at an end portion of the elongated body. In a particularly preferred implementation, microneedle device 400 is formed according to the teachings of the aforementioned US Patent Application Publication No. 2005/0209566 as an adapter for a syringe 406. For conciseness of presentation, the details of that implementation will not be repeated here.

As already mentioned, protective cover 404 is formed to as to function as a resilient clip configured to resiliently deform during motion from the closed state towards the open state. More specifically, at least part of device body 402 as shown has a generally round cross-sectional shape. Protective cover 404 is configured to circumscribe more than 180 degrees and less than 360 degrees, and more preferably between about 225 degrees and about 315 degrees, around device body 402 when in the closed state. The circumscribing of more than 180 degrees defines a gripping configuration which effectively retains the cover on the device body, while the incomplete circumscribing provides the capability for the clip to deform and open for attachment and removal. The extent of circumscribing together and the flexibility of the clip structure are the primary features defining the force required to attach and remove the cover.

Although this embodiment is conceptually somewhat similar to device 100 described above, the mechanical engagement which guides initial movement of the cover during removal is greatly simplified. In this case, the mechanical engagement is generated primarily by a projection 408 from device body 402 which engages a corresponding opening 410 formed in protective cover 404.

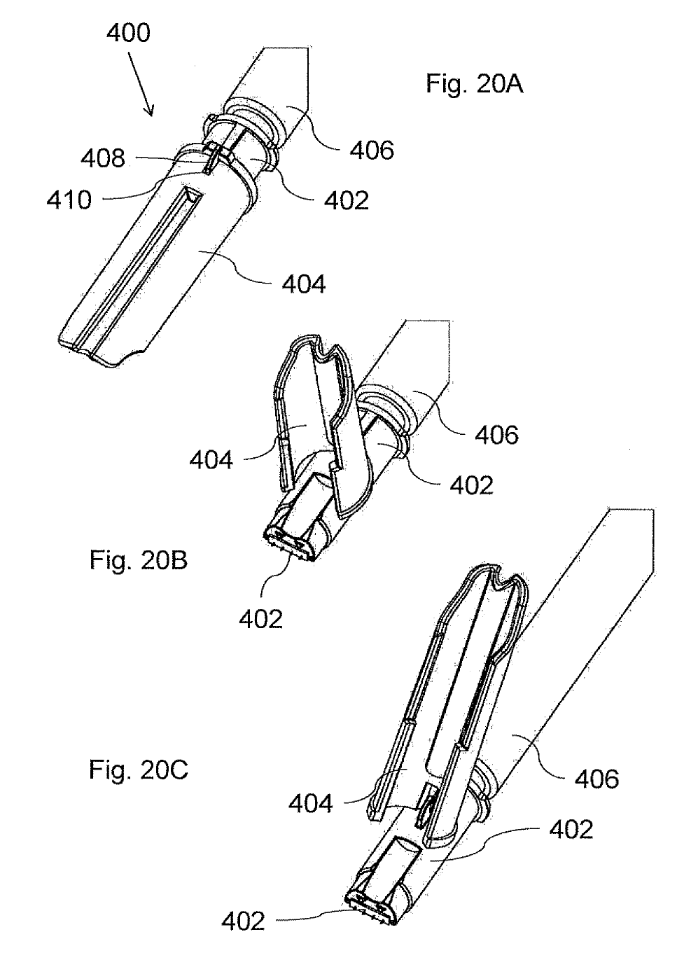

Details of the mechanical engagement are best seen in FIGS. 22A and 22B. Projection 408 is preferably formed so as to limit motion of protective cover 404 in various ways. Specifically, as seen in FIG. 22A, projection 408 is relatively narrow perpendicular to the length of the device and relatively long parallel to the length of the device. In other words, in a cross-section taken near the base of projection 408, the projection exhibits a major dimension parallel to the length and a minor dimension perpendicular to the length. Opening 410 is formed with a corresponding slot-like shape. These shapes cooperate to inhibit rotation of the protective cover relative to the device body about a radially-outward projecting axis, referred to as the direction of projection for projection 408.

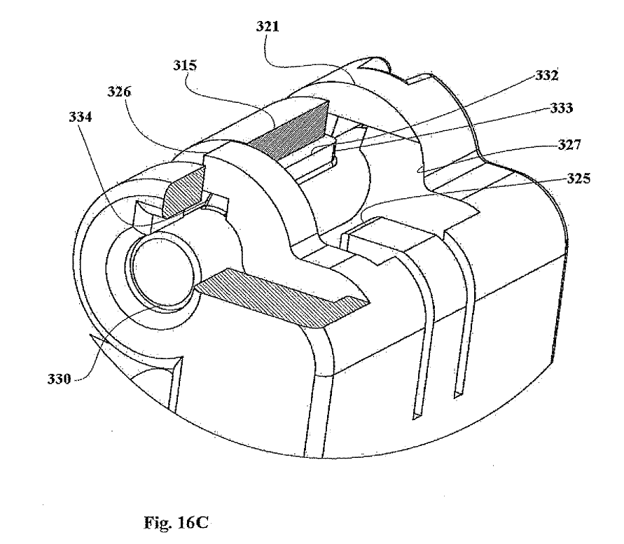

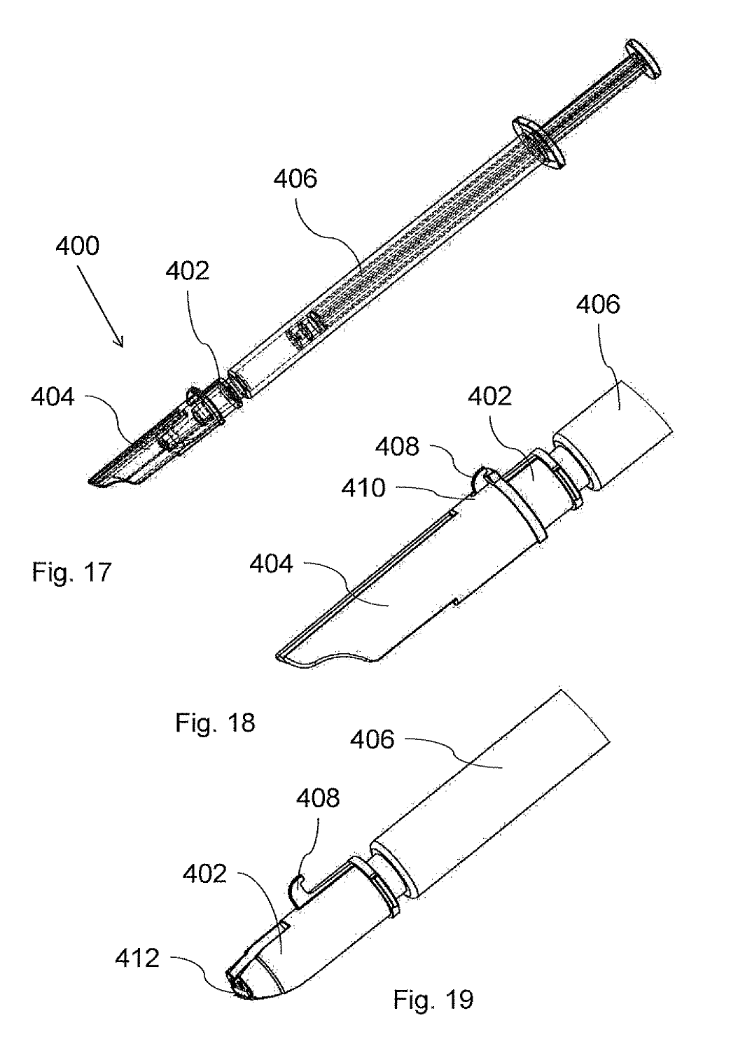

Additionally, as best seen in FIG. 22B, projection 408 includes an overhanging portion 409, extending in a direction away from the microneedle substrate surface, which gives a hook-shaped overall form to projection 408. The rear (proximal) part of cover 404 around the edge of opening 410 is formed as a reinforced retention element 411. The engagement of reinforced retention element 411 under overhanging portion 409 delimits a rotational motion of protective cover 404 away from the impact risk region as illustrated in FIGS. 20B-20C and 21B-21C. The length of opening 410 and the shape of projection 408 are configured to prevent premature disengagement of retention element 411 from overhanging portion 409 until protective cover 404 reaches a desired minimum angle of opening, typically in excess of about 45 degrees. The cover can then be disconnected through a rearward translation motion without risk of collision with the microneedles.

It will be noted that this and other embodiments of the present invention also provide an effective solution for safe recapping of the microneedle device. Specifically, replacement of the protective cover 404 can readily be performed by the reverse of the uncapping motion, namely, first engaging retention element 411 under overhanging portion 409 as shown in FIGS. 20C and 21C, and then pivoting the cover forwards and towards the device body 402 until it clips into position partially circumscribing the device body. The path of motion is thus controlled and all force applied by the user is generally in a forwards direction, i.e., from proximal to distal, thereby rendering the recapping acceptable from a safety point of view.

### Fifth Embodiment

### Side-Release Clip without Guide

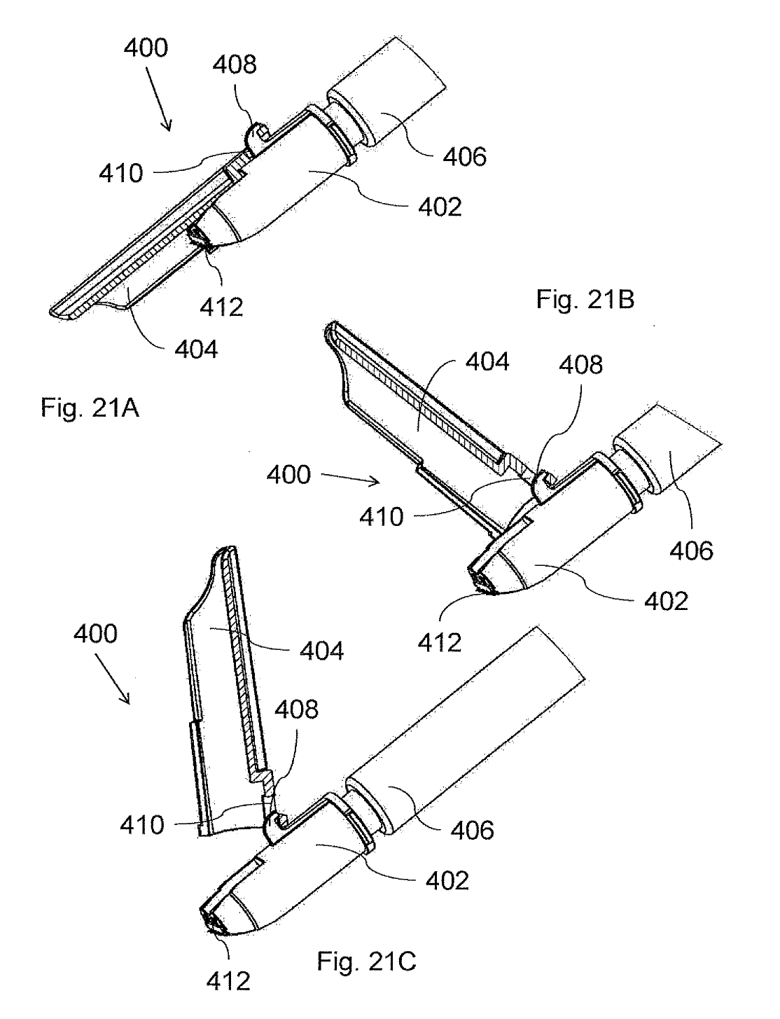

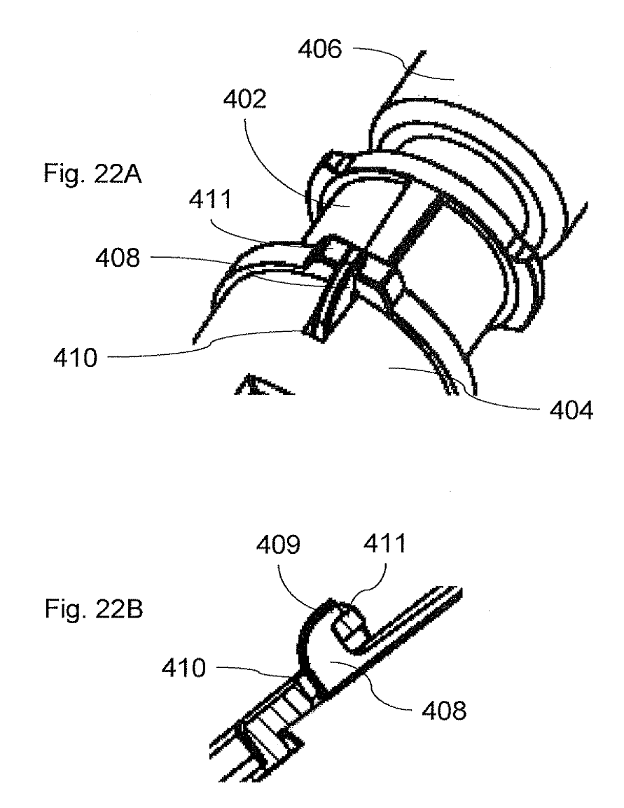

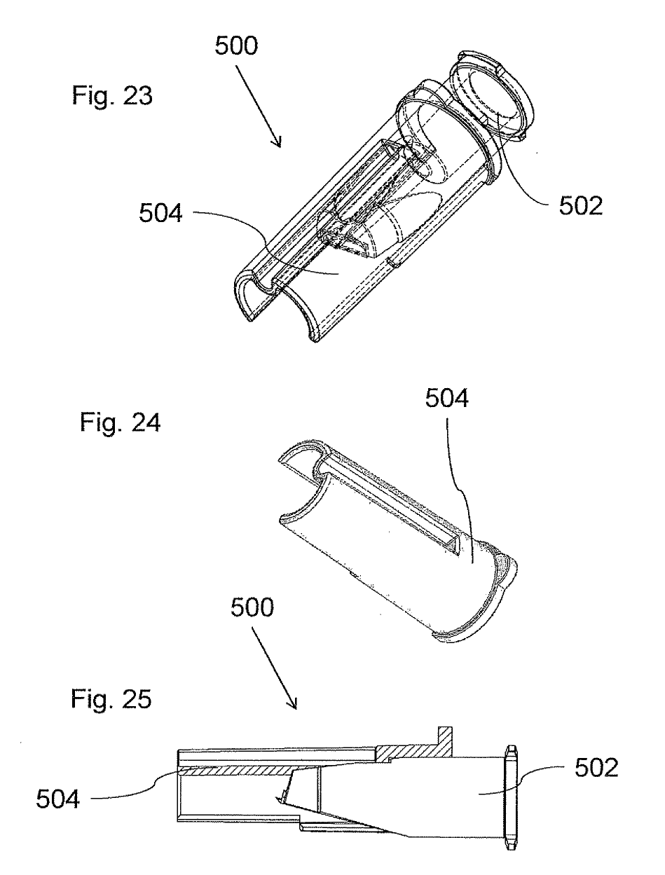

Turning finally to FIGS. 23-25, there is shown a fifth embodiment of a microneedle device, generally designated 500, constructed and operative according to the teachings of the present invention. This embodiment is essentially similar to the fourth embodiment but does not include projection 408 and opening 410. Although this embodiment lacks mechanical engagement delineating a specific path of motion for uncapping, the clip-like configuration facilitates and encourages motion from the closed state towards the open state in a direction non-parallel, and typically substantially transverse, to the length. Optionally, a ridge or step (not shown) formed on device body 502 engages complementary features on protective cover 504 in order to further inhibit removal of the cover by axial sliding displacement. Other corresponding geometrical shapes, such as the upper recessed slot shown here, may be provided for maintaining directional alignment between the cover and device body when the cover is engaged.

It will be appreciated that the above descriptions are intended only to serve as examples, and that many other embodiments are possible within the scope of the present invention as defined in the appended claims.

What is claimed is:

1. A microneedle device comprising:
(a) a device body including a microneedle substrate surface;
(b) at least one microneedle projecting from the microneedle substrate surface; and
(c) a protective cover deployable between a closed state in which said protective cover protects said at least one microneedle against inadvertent contact and an open state in which said at least one microneedle is exposed to facilitate bringing said at least one microneedle into functional engagement with a surface,
wherein said device body and said protective cover are configured such that, for at least part of a motion from said closed state towards said open state, said protective cover is guided by mechanical engagement with said device body configured to prevent impact between said protective cover and said at least one microneedle at least until said protective cover has cleared an impact risk region around said at least one microneedle.

2. The microneedle device of claim 1, wherein said device body is an elongated body having a length greater than each of two lateral dimensions, said microneedle substrate surface being located at an end portion of said elongated body, and wherein said protective cover includes a resilient clip configured to resiliently deform during motion from said closed state towards said open state.

3. The microneedle device of claim 2, wherein at least part of said device body has a generally round cross-sectional shape, and wherein said resilient clip is configured to circumscribe more than 180 degrees and less than 360 degrees around said device body when in said closed state.

4. The microneedle device of claim 3, wherein said resilient clip is configured to circumscribe between about 225 degrees and about 315 degrees around said device body when in said closed state.

5. The microneedle device of claim 2, wherein said mechanical engagement is generated at least in part by a projection from said device body engaged within a corresponding opening formed in said protective cover.

6. The microneedle device of claim 5, wherein said projection defines a direction of projection, and wherein said projection and said corresponding opening are configured to inhibit rotation of said protective cover relative to said device body about said direction of projection.

7. The microneedle device of claim 5, wherein a cross-section taken near a base of said projection has a major dimension parallel to said length and a minor dimension perpendicular to said length.

8. The microneedle device of claim 5, wherein said projection includes an overhanging portion extending in a direction away from said microneedle substrate surface, engagement of said corresponding opening with said overhanging portion delimiting a rotational motion of said protective cover away from said impact risk region.

9. The microneedle device of claim 1, wherein said mechanical engagement between said protective cover and said device body defines an axis of rotation for at least an initial part of a motion of said protective cover from said closed state towards said open state.

10. The microneedle device of claim 9, wherein said mechanical engagement is configured to allow translational displacement of said protective cover only after rotation through a predefined angle from said closed state.

11. The microneedle device of claim 10, wherein said mechanical engagement is further configured to define a path of said translational displacement.

12. The microneedle device of claim 9, wherein said mechanical engagement comprises a permanent hinged interconnection between said protective cover and said device body about said axis of rotation.

13. The microneedle device of claim 12, wherein said permanent hinged interconnection is further configured to allow displacement of said protective cover once from said closed state to said open state and, after returning to said closed state, to lock so as to prevent subsequent displacement to said open state.

14. The microneedle device of claim 1, further comprising a recapping lock mechanism configured to allow displacement of said protective cover once from said closed state to said open state and, after returning to said closed state, to lock so as to prevent subsequent displacement to said open state.

15. The microneedle device of claim 1, wherein a normal to a plane of said microneedle substrate surface defines a reference direction, and wherein said mechanical engagement defines a direction of linear displacement substantially transverse relative to said reference direction.

16. The microneedle device of claim 1, wherein said at least one microneedle is implemented as a linear array of microneedles.

17. The microneedle device of claim 16, wherein said linear array is deployed parallel to, and substantially adjacent to, an edge of said device body.

18. A microneedle device comprising:
(a) an elongated device body having a length greater than each of two lateral dimensions, said device body including a microneedle substrate surface located at an end portion of said device body;
(b) at least one microneedle projecting from the microneedle substrate surface; and
(c) a protective cover deployable between a closed state in which said protective cover protects said at least one microneedle against inadvertent contact and an open state in which said at least one microneedle is exposed to facilitate bringing said at least one microneedle into functional engagement with a surface, said protective cover including a resilient clip configured to resiliently deform to allow motion from said closed state towards said open state in a direction non-parallel to said length.

19. The microneedle device of claim 18, wherein at least part of said device body has a generally round cross-sectional shape, and wherein said resilient clip is configured to circumscribe more than 180 degrees and less than 360 degrees around said device body when in said closed state.

20. The microneedle device of claim 19, wherein said resilient clip in configured to circumscribe between about 225 degrees and about 315 degrees around said device body when in said closed state.

21. The microneedle device of claim 18, wherein said device body and said protective cover are configured such that, for at least part of a motion from said closed state towards said open state, said protective cover is guided by mechanical engagement with said device body configured to prevent impact between said protective cover and said at least one microneedle at least until said protective cover has cleared an impact risk region around said at least one microneedle.

22. The microneedle device of claim 18, wherein said device body includes a lateral projection and said protective cover features a corresponding opening, said corresponding opening being engaged with said lateral projection in said closed state so as to at least partially delimit a path of motion from said closed state towards said open state.

23. The microneedle device of claim 22, wherein said projection defines a direction of projection, and wherein said projection and said corresponding opening are configured to inhibit rotation of said protective cover relative to said device body about said direction of projection.

24. The microneedle device of claim 22, wherein a cross-section taken near a base of said projection has a major dimension parallel to said length and a minor dimension perpendicular to said length.

25. The microneedle device of claim 22, wherein said projection includes an overhanging portion extending in a direction away from said microneedle substrate surface, engagement of said corresponding opening with said overhanging portion delimiting a rotational motion of said protective cover away from said microneedle substrate surface.

26. A method of using a microneedle device comprising the steps of:
(a) providing a microneedle device including:
(i) an elongated device body having a length greater than each of two lateral dimensions, the body including a microneedle substrate surface located at an end portion of said elongated body,
(ii) at least one microneedle projecting from the microneedle substrate surface, and
(iii) a protective cover deployed so as to protect said at least one microneedle against inadvertent contact; and
(b) displacing said protective cover so as to expose said at least one microneedle for functional engagement with a surface,
wherein said displacing is performed in a direction non-parallel to said length.

27. The method of claim 26, wherein said displacing is performed in a direction generally transverse to said length.

28. The method of claim 26, wherein said displacing is performed in a generally pivotal motion.

29. The method of claim 26, wherein device body and said protective cover have features for mechanical engagement such that at least an initial part of said displacing occurs along a path of motion delineated by said features for mechanical engagement.
